(19) United States
(12) Patent Application Publication
(10) Pub. No.: US 2014/0187627 A1
(43) Pub. Date: Jul. 3, 2014

(54) USES OF C15-SUBSTITUTED ANDROGRAPHOLIDE DERIVATIVES IN THE PREPARATION OF ANTI-HEPATITIS B VIRUS MEDICAMENT

(71) Applicants: Guifu DAI, Henan (CN); Haiwei XU, Henan (CN); Zhiwen JIANG, Henan (CN); Hongmin LIU, Henan (CN); Yanan WANG, Henan (CN); Kuicheng ZHU, Henan (CN); Zhenwei WU, Henan (CN); Weiyi LI, Henan (CN); Fengjuan WU, Henan (CN); Mengjiao LIU, Henan (CN); Wei HAN, Henan (CN)

(72) Inventors: Guifu DAI, Henan (CN); Haiwei XU, Henan (CN); Zhiwen JIANG, Henan (CN); Hongmin LIU, Henan (CN); Yanan WANG, Henan (CN); Kuicheng ZHU, Henan (CN); Zhenwei WU, Henan (CN); Weiyi LI, Henan (CN); Fengjuan WU, Henan (CN); Mengjiao LIU, Henan (CN); Wei HAN, Henan (CN)

(73) Assignee: ZHENGZHOU UNIVERSITY, Henan (CN)

(21) Appl. No.: 14/130,695

(22) PCT Filed: Jul. 3, 2012
(86) PCT No.: PCT/CN2012/078083

§ 371 Date: Mar. 20, 2014

(30) Foreign Application Priority Data
Jul. 4, 2011 (CN) 201110184294.4

(51) Int. Cl. C07D 307/33; A61K 31/365

(52) U.S. Cl. CPC C07D 307/33; A61K 31/365
USPC 514/473

(57) ABSTRACT

Disclosed is a use of C15-substituted andrographolide derivatives in preparation of anti-hepatitis B virus medicaments. In the present invention, the HepG2.2.15 cells are used to measure the amount of the hepatitis B virus surface antigen (HBsAg) secretion in the supernatant of the culture; the duck hepatitis B virus (DHBV) is used to infect the model and the DHBV-DNA level in serum is measured, and the pathological change in hepatic tissue is observed. A number of andrographolide derivative compounds are screened, compounds having a good anti-HBV effect are preferred, which has a structure represented by general formula 1 set forth herein. Due to high anti-HBV activity and low toxicity, as well as good protection against hepatic injury, the compounds can be used as the active ingredient for preparing anti-HBV medicaments, thereby providing a new pharmaceutical way for treatment of hepatitis, and broadening the range of clinical medicines.

### FIELD OF THE INVENTION

The present invention relates to the pharmaceutical use of andrographolide derivatives, specifically the use of the C (15)-substituted andrographolide derivatives in the preparation of anti-hepatitis B drugs, which belongs to the field of pharmaceutical chemistry.

### BACKGROUND OF THE INVENTION

Andrographolide (AD), a major active diterpenoid lactone compound of Andrographis paniculata, is an essential active ingredient of traditional Chinese herb medicine Andrographis, which has been mainly used in clinical to treat some infectious diseases, including upper respiratory tract infections, bacillary dysentery and so on. Anti-tumor, hepatoprotection and anti-virus activities of AD have been proved recently. The anti-virus effects of Andrographis paniculata and its extract have also been widely reported.

The extract of Andrographis paniculata is very effective at treating respiratory tract infections and viral pneumonia, and reducing the prevalence and intensity of symptoms associated with the common cold; total flavonoids in Andrographis paniculata combined with AD or its derivative could inhibit the influenza virus and adenovirus infection, and delay the progression of herpes virus infection (CN: 200610080719.6). The effectiveness of AD and its derivatives against infections by flavivirus, pestivirus or hepatitis C virus (HCV) (CN: 200580046253.1) and severe acute respiratory syndromes (SARS) (CN: 03129127.9) have been reported. The combination of ingredients of Andrographis paniculata and another plant or its components in combination have been proved to be effective to virus. According to the U.S. Pat. No. 5,833,994, hydrocarbon receptor ligands and andrographolide in combination can be used in the treatment of viral infections. Dehydroandrographolide succinic acid monoester partially interfered with HIV-induced cell fusion and with the binding of HIV to the H9 cell. HIV-1 replication in vitro was reported to be inhibited by the methanol extract of Andrographis paniculata by inhibiting c-Mos. However, it were also reported that aqueous extracts of Andrographis paniculata had little or even no inhibitory effect on HIV-1; extract of Andrographis paniculata did not remarkably inhibit the expression of surface antigen of HBV.

Hepatitis B virus (HBV), a member of hepadnaviradae, is the pathogen of hepatitis B. More than 350 million people worldwide have been infected with HBV, and about 120 million people suffer persistent infection caused by HBV in China. Therefore, there is a huge market demand for anti-HBV drugs. So far, anti-HBV drugs are mainly nucleoside analogs, such as Lamivudine, and immunomodulatory agents, such as interferon. However, adverse reactions, drug resistance and rebound after drug withdrawal often occur in clinical use.

The synthesis of 15-alkylidene substituted andrographolides has been reported in patent CN: 200510107247.4, which is the previous research of the inventors. Subsequently, 15 -p-chlorobenzylidene-14-deoxy-11, 12-didehydro-3, 19-dinicotinate-andrographolide, an α-glucosidase inhibitor (CN: 200810231375.3), has been proved having anti-HBV effect in vitro. Therefore, further studies to develop more effective anti-HBV drugs based on derivatives of andrographolide have been made, which is of great significance for expanding the usage of this kind of compounds.

### SUMMARY OF THE INVENTION

Based on the previous research, through extensive screening, the inventors found that andrographolide derivatives, with the molecular structure shown in formula 1, showed remarkable anti-HBV activity. The aim of this invention is to provide the application of these compounds, particularly relates to the application in preparing anti-hepatitis drugs.

Compounds of the invention having the structure formula 1:

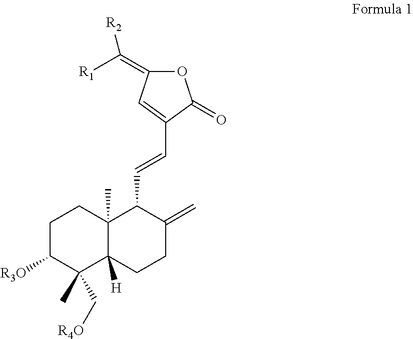

wherein R₁ is hydrogen; R₂ is phenyl, 4-fluorophenyl, 4-chlorophenyl, 4-bromophenyl, 3-fluorophenyl, 3-chlorophenyl, 3-bromophenyl, methoxyphenyl or polymethoxyphenyl; R₃ and R₄ are each independently hydrogen or COR₅, of which R₅ is 3-pyridyl; but when R₂ is 4-chlorophenyl, neither R₃ nor R₄ is COR₅.

Preferred compounds of this invention are those wherein R₁ is hydrogen; R₂ is phenyl, 4-fluorophenyl, 4-chlorophenyl, 4-bromophenyl, 3-fluorophenyl, 3-chlorophenyl, 3-bromophenyl or 3-methoxyphenyl, 4-methoxyphenyl; R₃ and R₄ are both hydrogen.

Preferred compounds of this invention are also those wherein R₁ is hydrogen; R₂ is phenyl, 4-fluorophenyl, 4-Bromophenyl, 3-fluorophenyl, 3-chlorophenyl, 3-bromophenyl, 3-methoxyphenyl or 4-methoxyphenyl; R₃ and R₄ are both COR₅, of which R₅ is 3-pyridyl.

Further preferred compounds are as follows:
A: R₁=H, R₂=4-Cl—C₆H₄, R₃=R₄=H;
B: R₁=H, R₂=C₆H₅, R₃=R₄=H;
C: R₁=H, R₂=4-F—C₆H₄, R₃=R₄=H;
D: R₁=H, R₂=4-F—C₆H₄, R₃=R₄=COR₅, R₅=C₅H₄N;
E: R₁=H, R₂=4-Br—C₆H₄, R₃=R₄=H;
F: R₁=H, R₂=3-Br—C₆H₄, R₃=R₄=COR₅, R₅=C₅H₄N;
G: R₁=H, R₂=2,4,5-triMeO—C₆H₂, R₃=R₄=H;

The preparation method used to synthesize the compounds of this invention mentioned above has been made public in the previous patent (CN: 200510107247.4) and in the paper (BMC 2007; Xu H W, et al.), which is as follows in brief: One of the 14-deoxy-11,12-didehydroandrographolide or 3,19-ester derivatives of 14-deoxy-11,12-didehydroandrographolide and a kind of aldehydes were dissolved in methanol, ethanol or tetrahydrofuran, and then the andrographolide derivatives showed in Formula 1 can be obtained through heating the mixture at the temperature of 15 to 70° C., catalyzed by base at 0.2 to 5% (mol/mol).

Wherein the aldehydes used are one of the benzaldehyde, halogenated benzaldehyde, p-methoxybenzaldehyde and so on; the further optimized halogenated benzaldehydes are p-fluoro-, p-chloro- and p-bromobenzaldehyde; the ester derivatives of 14 -deoxy-11, 12-didehydro-andrographolide used are those when R₃ and R₄ in formula 1 are both COR₅, of which R₅ is 3-pyridyl.

To achieve the objects of the present invention, HBV-transfected HepG2.2.15 cell line was used to investigate the in vitro anti-HBV activity of C (15)-substituted andrographolide derivatives, and the duck hepatitis B virus (DHBV) infected ducklings are used to study the in vivo anti-DHBV effects. Proved by the experiments, compounds mentioned above had remarkably inhibitory effects on HBV (DHBV), which has potential to be used in preparation of anti-HBV drugs. According to the conventional pharmaceutical methods and process, the derivatives can be used as an active ingredient, or in combination with another drug, and mixed with a pharmaceutically acceptable auxiliary and/or additive, to prepare an anti-hepatitis B drug in an oral or injectable preparation. The oral preparation is tablet, pill, capsule, granule or syrup; the injectable preparation is an injection or a lyophilized powder for injection.

The advantages of the present invention include: these compounds are of high efficiency and low toxicity, and have potential to be used as the active pharmaceutical ingredients in the treatment and prevention of hepatitis B, which would provide a new path for developing anti-HBV drugs and expand the optional range of clinical drugs.

### BRIEF DESCRIPTION OF THE FIGURES

FIG. 1 shows the in vitro inhibitory activities of derivatives A-G of the present invention against HBsAg secretion; the IC₅₀ value of Lamivudine is approximately 10 μg/ml (43.67 μmol/L) after treatment for 9d.

FIG. 2 shows the therapeutic index (TI) of derivatives A-G of the present invention to HBV in HepG2.2.15 cells; TI value of Lamivudine is greater than 2 while treated for 9d.

FIG. 3 shows the in vivo anti-DHBV effect of derivatives A-G of the present invention at the dosage of 0.35 mmol/kg (with 5d treatment); Lamivudine (3TC) is administered at the dose of 20 mg/kg; compared with control group, *P<0.05, **P<0.01.

FIG. 4 shows the in vivo anti-DHBV effect of derivative A of the present invention; positive drug Lamivudine (3TC) is administered at the dose of 20 mg per kg of body weight; compared with control group, *P<0.05, **P<0.01.

FIG. 5 shows the histopathological changes in duck livers of DHBV-infected ducks administrated with derivative A; picture A represents normal group, picture B represents model group, picture C represents positive drug group, picture D and picture E represents respectively the low dose and high dose group of derivative A.

### DETAILED DESCRIPTION OF THE INVENTION

Compound A-G was synthesized with the method mentioned above.

Compound A: R₁=H, R₂=4-Cl—C₆H₄, R₃=R₄=H; IR 3413, 2934, 1749, 1632, 1490, 1442, 1090, 1035, 891 cm⁻¹; ¹H NMR (400 MHz, CDCl₃) 7.77(2H, d, J=6.8 Hz), 7.49(1H, m), 7.40(3H, m), 6.86(1H, dd.J=10.0, 15.6 Hz), 6.24(1H, d, J=16.0 Hz), 6.17(1H, s), 4.76(1H, s), 4.48(1H, s), 4.03(1H, d, J=10.8 Hz), 3.25(1H, d, J=10.8 Hz), 3.31(1H, t, J=7.2 Hz), 2.41(2H, m), 2.03(1H, m), 1.78(1H, m), 1.64(2H, m), 1.44(1H, m), 1.35(1H, 1H, m), 1.23(2H, m), 1017(3H, s), 0.82(3H, s); ¹³CNMR (100 MHz, CDCl₃): δ 168.4, 148.9, 148.1, 137.7, 136.5, 131.7, 131.2, 129.9, 126.9, 121.5, 131.5, 108.8, 79.5, 63.4, 61.6, 5434, 42.7, 38.8, 38.4, 36.6, 28.1, 23.2, 15.9; HR-MS m/z: [M+Na]+, 461.2130, (calcd. 461.2104).

Compound B: R₁=H, R₂=C₆H₅, R₃=R₄=H; IR 3393, 2933, 2847, 1750, 1644, 1450, 1036, 942, 894, 758, 690 cm⁻¹; ¹H NMR (400 MHz, CDCl₃) 7.77(2H, d, J=7.6 Hz), 7.40(2H, m), 7.32(1H, m), 7.12(1H, s), 6.92(1H, dd, J=10.0, 15.7 Hz), 6.20(1H, d, J=15.7 Hz), 5.96(1H, s), 4.80(1H, s), 4.54(1H, s), 4.24(1H, bs), 3.49(1H, bs), 3.38(1H, bs), 2.46(1H, d, J=13.4 Hz), 2.36(1H, d, J=10.0 Hz), 2.27(2H, bs), 2.05(1H, t, J=13.0 Hz), 1.8(3H, m), 1.54(1H, J=13.0 Hz), 1.41(1H, m), 1.38(3H, s), 1.14(2H, m), 0.84(3H, s); ¹³CNMR (100 MHz, CDCl₃): δ 168.8, 148.0, 147.5, 137.6, 135.5, 133.2, 130.4, 128.9, 128.8, 127.0, 121.5, 133.1, 109.3, 80.8, 64.2, 61.9, 54.6, 13.0, 38.7, 38.3, 36.5, 28.1, 22.9, 22.8, 15.9; HR-MS m/z: [M+Na]+, 443.2187, (calcd. 443.2199).

Compound C: R₁=H, R₂=4-F—C₆H₄, R₃=R₄=H; IR 3293, 3081, 2944, 2849, 1747, 1642, 1600, 1507, 1449, 1418, 1232, 1362, 1038, 986, 943, 989 cm⁻¹; ¹H NMR (400 MHz, CDCl₃) 7.80(2H, m), 7.73(1H, s), 7.30(2H, m), 6.83(1H, dd, J=10.1, 15.8 Hz), 6.35(1H, s), 6.27(1H, d, 15.8 Hz), 5.05(1H, bs), 4.75(1H, s), 4.45(1H, s), 4.1(1H, bs), 3.86 (1H, d, J=10.9), 3.30(1H, d, J=13.0 Hz), 3.23(1H, m), 2.43(1H, d, J=10.1 Hz), 2.38(1H, br), 2.0(1H, m), 1.71(1H, br), 1.59(2H, m), 1.38(1H, m), 1.34(1H, m), 1.20(2H, m), 1.10(3H, s), 0.79(3H, s). ¹³CNMR (100 MHz, CDCl₃): δ 168.6, 163.4, 163.4, 160.9, 148.9, 147.3, 137.2, 132.6, 132.5, 130.1, 126.2, 121.5, 136.3, 136.1, 131.8, 108.4, 78.8, 62.9, 60.9, 53.9, 42.6, 38.7, 38.2, 36.4, 27.8, 23.3, 23.2, 15.7; HR-MS m/z: [M+Na]+461.2130 (calcd. 461.2104).

Compound D: R₁=H, R₂=4-F—C₆H₄, R₃=R₄=COR₅, R₅=C₅H₄N; IR: 3440.3, 2938.6, 2849.1, 1763.8, 1716.9, 1639.9, 1593.7, 1465.9, 1287.9, 1248.1, 1194.5, 1115.6, 1027.9, 743.3 cm⁻¹; ¹H NMR (400 MHz, CDCl₃): δ 9.15(2H, br), 8.75(2H, br), 8.24(2H, m), 7.73(2H, d, J=8.8 Hz), 7.39(1H, br), 7.26(1H, br), 7.12(1H, s), 7.02(1H, dd, J=10.0, 15.6 Hz), 6.95(2H, d, J=8.8 Hz), 6.27(1H, d, J=15.4 Hz), 6.02(1H, s), 5.02(1H, t, J=8.04), 4.85(2H, om), 4.62˜4.56(2H, om), 2.55(1H, d, J=13.3 Hz), 2.46(1H, d, J=10.0 Hz), 2.13(1H, br), 1.98(1H, br), 1.89(2H, br), 1.68(2H, om), 1.52(1H, m), 1.39(1H, m), 1.25(3H, s), 1.01(3H, s).

Compound E: R₁=H, R₂=4-Br—C₆H₄, R₃=R₄=H; IR 3291, 2927, 2851, 1747, 1642, 1603, 1510, 1457, 1257, 1377, 1035, 941, 900 cm⁻¹; ¹H NMR (400 MHz, CDCl₃) 7.73(2H, d, J=8.8 Hz), 7.10(1H, s), 6.92(2H, d, J=8.8 Hz), 6.87(1H, dd, J=10.1, 15.8 Hz), 6.19(1H, d, J=15.8 Hz), 5.92(1H, s), 4.79(1H, d, J=0.89 Hz), 4.55(1H, d, J=0.89 Hz), 4.22(1H, d, J=13.0 Hz), 3.48(1H, m), 3.35(1H, d, J=13.0 Hz), 2.45(1H, m), 2.35(1H, d, J=10.0 Hz), 2.04(1H, m), 1.79(2H, m), 1.74(1H, m), 1.54(1H, m), 1.34(1H, m), 1.27(3H, s), 1.24(1H, m), 1.15(1H, m), 0.84(3H, s); ¹³CNMR (100 MHz, CDCl₃): δ 169.0, 160.2, 148.1, 146.2, 136.8, 135.6, 132.2, 126.2, 125.9, 121.6, 134.4, 133.1, 109.3, 64.2, 61.9, 55.3, 54.7, 43.0, 38.7, 38.3, 36.6, 28.1, 22.9, 22.6, 15.9.

Compound F: R₁=H, R₂=3-Br—C₆H₄, R₃=R₄=COR₅, R₅=C₅H₄N IR:3426, 2929, 2853, 1768, 1720, 1640, 1591, 1473, 1421, 1285, 1117, 990, 895, 742, 700 cm⁻¹; ¹H NMR (400 MHz, CDCl₃): δ 9.14(2H, br), 8.73(2H, br), 8.26(1H, d, J=8.8 Hz), 8.22(1H, d, J=8.0 Hz), 7.86(1H, s), 7.73(1H, d, J=8.0 Hz), 7.43(1H, m), 7.38(1H, m), 7.29(1H, d, J=7.9 Hz), 7.24(1H, m), 7.13(1H, s), 6.90(1H, dd, J=10.0, 15.6 Hz), 6.27(1H, d, J=15.6 Hz), 5.89(1H, s), 5.02(1H, t, J=8.0 Hz), 4.86(2H, ol), 4.61(1H, s), 4.57(1H, m), 2.55(1H, br), 2.48(1H, d, J=10.0 Hz), 2.16(1H, br), 2.02(1H, br), 1.91(2H, br), 1.71(2H, ol), 1.52(1H, d, J=11.4 Hz), 1.29(1H, m), 1.25(3H, s), 1.02(3H, s); ¹³C NMR (100.6 MHz, CDCl₃): δ:168.4, 165.1, 164.7, 153.2, 150.5, 148.3, 147.4, 137.7, 137.5, 135.6, 135.3, 132.9, 131.7, 131.4, 130.5, 126.2, 123.5, 121.9, 111.4, 109.9, 81.2, 65.5, 61.9, 54.9, 42.2, 38.9, 38.3, 36.6, 29.7, 24.4, 23.9, 22.8, 15.5; HRMS m/z: (M+H+)709.1918 (calcd. 709.1913).

Compound G: R₁=H, R₂=2,4,5-triMeO—C₆H₂, R₃=R₄=H; IR 3431, 2936, 2845, 1760, 1643, 1578, 1505, 1455, 1422, 1335, 1250, 1128, 1033, 892; ¹H NMR (400 MHz, CDCl₃) δ 7.1(1H, s), 7.01(2H, br), 6.90(1H, dd, J=10.1, 15.6 Hz), 6.22(1H, d, J=15.6 Hz), 5.88(1H, s), 4.80(1H, s), 4.54(1H, s), 4.22(1H, d, J=10.7 Hz), 3.90(9H, om), 3.48(1H, m), 3.35(1H, d, J=10.8 Hz), 2.45(1H, d, J=13.2 Hz), 2.35(1H, d, J=10.1 Hz), 2.01(1H, br), 1.82˜1.74(3H, om), 1.53(1H, d, J=13.2 Hz), 1.38(1H, m), 1.27(3H, s), 1.24˜1.13(2H, om), 0.84(1H, s); ¹³CNMR (100 MHz, CDCl₃): δ 168.7, 153.2, 148.0, 147.1, 137.5, 135.3, 128.8, 126.6, 121.4, 114.8, 113.0, 109.3, 107.7, 106.5, 80.8, 64.1, 61.8, 60.9, 56.3, 56.2, 54.6, 43.0, 38.7, 38.3, 36.5, 28.1, 22.9, 22.6, 15.9, 14.1; HR-MS m/z: [M+Na]+533.2519, (calcd. 533.2515).

Compounds A-G were used as examples to illustrate the anti-HBV activity in detail by pharmacological test.

### Example 1

### In Vitro Anti-HBV Activity of Andrographolide Derivatives

### 1. Cell Culture and Compound Treatment

HepG2.2.15 cell line, stably transfected with HBV-DNA, was used to evaluate the inhibition of the compounds against the HBV surface antigen (HBsAg) level in the culture supernatant of HepG2.2.15 cell. HepG2.2.15 cells were seeded at a density of 1.25×10⁴ cells/well into 48-well plates, 0.5 ml RPMI1640 medium containing 10% fetal bovine serum, 100 unit/ml penicillin, 100 μg/ml streptomycin and 380 μg/ml geneticin (G418) was added to the well. Cells were incubated at 37° C. in a humidified incubator of 5% CO₂ for 24 h, and then the culture medium was replaced by fresh medium containing Lamivudine (the positive drug) and compounds of this invention at five concentrations. The culture medium was replaced by a fresh one after 3 days and 6 days incubation, respectively. On d9, the supernatant was collected to determine the HBsAg level and the cells were used to assay the viability by MTT method.

### 2. Cytotoxicity Evaluation by MTT Assay

Add 100 μl PBS solution containing 0.5 mg/ml MTT [3-(4,5-dimethylthiazol-2-yl)-2,5diphenyltetrazolium bromide] to the well after removing the supernatant, incubate at 37° C. for 4 h, remove the medium, add 00 μL of DMSO to each well and the plates were shaken for 10 min. The optical density value (A) of each well was read using a PowerWaveX Microplate Scanning Spectrophotometer (Bio-tek Instruments, Inc) at 490 nm. Cells survival rate was calculated.

### 3. Determination of HBsAg by ELISA

Diagnostic kit of HBsAg (commercially available product) was used in the detection of HBsAg according to the manufacturer's direction. The optical density values of each well at 450 nm and 630 nm (reference wavelength) were measured using a PowerWaveX Microplate Scanning Spectrophotometer (Bio-tek Instruments, USA), then the inhibition rate and the therapeutic index (TI) were calculated as follows.

Inhibition rate(%)=(OD_{control}−ODₛₐₘₚₗₑ)33 100%/OD_{control}; TI value=TC₅₀/IC₅₀, wherein TC₅₀ means the median toxic concentration, IC₅₀ means the median effective concentration. TI≧2 indicated that the compound was of high efficiency and low toxicity.

### 4. Activity Results

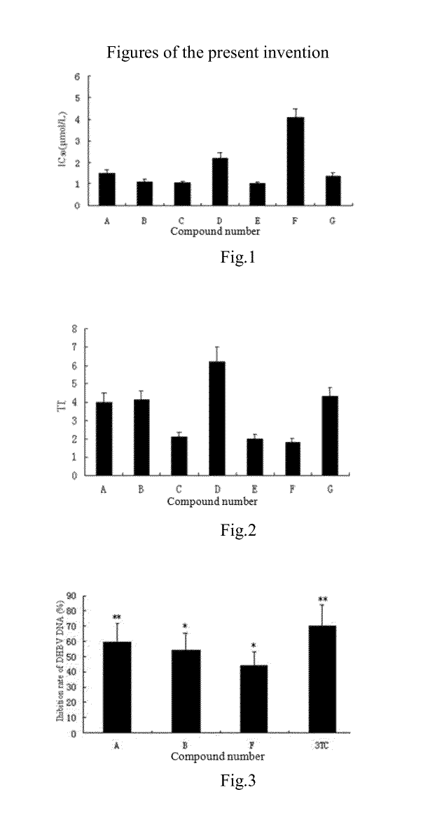

Derivatives A-G of the resent invention were found to have significant inhibitory effect on the secretion of HBsAg of HepG2.2.15 cell in a time-and dose-dependent manner, by screening large numbers of andrographolide derivatives. The IC₅₀ values of the compounds of the present invention were lower than 4.0 μmmol/l (FIG. 1); whereas the IC₅₀ value of positive drug Lamivudine was approximately 10 μg/ml (43.67 μmol/l).

The TI values of the compounds, except for compound F, were greater than 2, as shown in FIG. 2, which indicated that the compounds were of high efficiency and low toxicity.

### Example 2

### In Vivo Anti-DHBV Activities of Andrographolide Derivatives

### 1. Animals and Materials

Cherry Valley ducks, male, were obtained from a commercial hatchery; DHBV DNA positive serum were collected and preserved at −80 ° C.

### 2. Equipment, Drugs and Reagents

The LightCycler® Real-Time PCR Systems (Roche Applied Science), UnoII Thermocycler (Biometra, Germany), Milli-QB.S Ultrapure Water System (Millipore Limited Company, USA), LEICA RM2235 rotary microtome (Leica Biosystems, Germany), YD-A intelligent biological tissue spreading machine, YD-B intelligent biological tissue drying machine and YD-6D automatic tissue embedding machine (Yidi Medical Appliance Factory, Jinhua, Zhejiang). Compound A-G of the present invention was synthesized by the applicant; Lamivudine was commercially available. The above-mentioned experimental drugs were prepared with normal saline emulsified with Tween-80 (0.1% v/v) and dispersed in carboxymethyl cellulose (0.5% v/v). SYBR Green I was provided by TaKaRa Biotechnology (Dalian) Co., Ltd. The forward and reverse primers were synthesized by Sangon (Shanghai) Co., Ltd.

### 3. Experimental Methods

200 μl blood was collected form jugular vein of one-day-old Cherry Valley ducks, then serum was separated and DNA was extracted. Ducklings which were not congenitally DHBV-infected and were consistent in body condition were screened out by PCR method for experiment. Ducklings were inoculated intravenously via the shin vein with DHBV-DNA-positive serum (0.2 ml/animal) at the age of 3-day-old.

Seven days later, collected blood via jugular vein, separated serum, examine the DHBV DNA level in serum of ducks by PCR assay. The DHBV-infected ducklings, were randomly divided into groups and the drugs were orally administered once a day (1 ml/200 g body weight).

Firstly, compounds with representative structures were selected to make initial screening. Blood samples were collected via the jugular vein 5 days after treatment, serum was separated, and the copies of DHBV DNA were measured to calculate the inhibition rate. Compound A with good activity was selected for the two-week administration (1 time/day) evaluation. Blood samples were separately collected from the jugular vein 7 day, 14 day after treatment, and 5 days after cessation of treatment, serum was separated, and the copies of DHBV DNA were measured to calculate the inhibition percentage. Half of ducklings were killed 14 d after treatment. Duckling's liver were removed, fixed in 4% paraformaldehyde solution, and stained using hematoxylin and eosin dye. The serum DHBV DNA levels were measured by Real-time PCR (SYBRGreen I) method [Journal of Yangzhou University Natural Science Edition; 2010, 31 (3)]. Percentages of the decrease by drugs in DHBV DNA level was calculated using the formula % decrease in DHBV DNA level=Cv−Cd/Cv×100, where Cv is mean serum DHBV DNA copies in ducklings administrated with vehicle and Cd is mean serum DHBV DNA copies in ducklings administrated with drugs

Statistical analysis was performed using the SPSS17.0 software. The results were expressed as mean±SD and a p value of 0.05 was regarded as significant.

### 4. Activity Results:

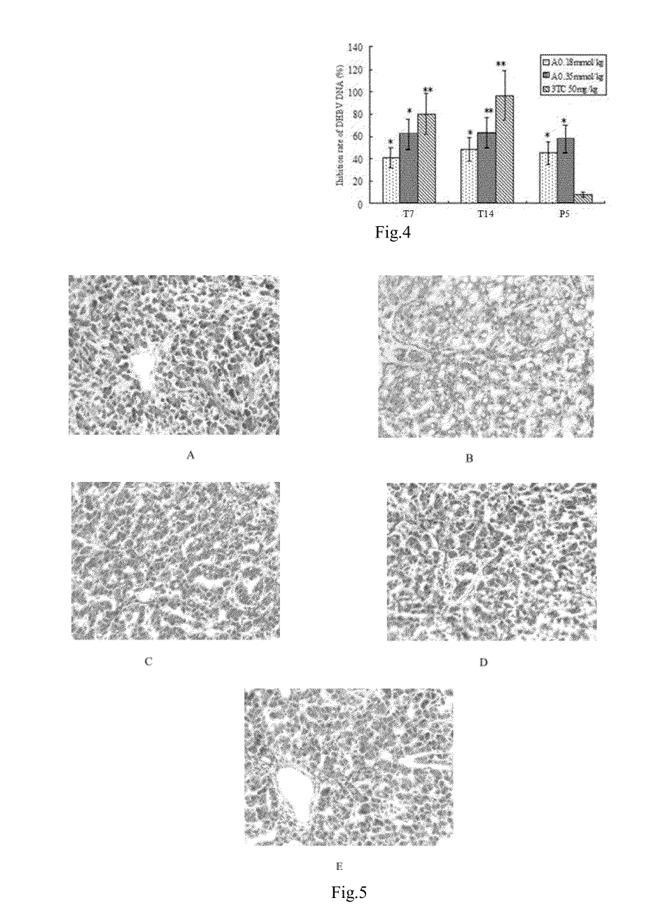

Compounds, shown in FIG. 3 and FIG. 4, significantly reduced the serum DHBV-DNA levels in ducklings after 5 day administration, compared with those in ducklings administrated with vehicle (P<0.05), of which 57.8% decreasing by compound A being observed (P<0.01). Therefore, it has been proved that compounds of the present invention are effective to inhibit DHBV DNA replication in vivo.

Showed by the serum DHBV DNA level in ducking treated with compound after 7 day and 14 day, and after treatment cessation for 5 days, compound A at both high and low dose significantly lowered the serum DHBV-DNA level, compared with the vehicle (FIG. 4). The results also showed that the inhibition on DHBV DNA by compound A had sustained, while that by Lamivudine had lost since 5 days after the treatment cessation, though Lamivudine at 20 mg/kg (FIG. 3) and 50 mg/kg dose (FIG. 4) had strongly inhibition on DHBV-DNA during the administration period.

### Example 3

### The Limited Toxicity Test of Compounds A-G

### 1. Animals

Kunming mice of clean grade, weighing 20±2 g, half male and half female, were purchased from Animal Experiment Center of Henan Province. Certificate of Quality No. 0009898.

### 2. Drugs

Compounds A-G of the present invention.

### 3. Experimental Methods

The mice were divided into groups randomly, then were intragastrically administrated with one of compounds A-G at the dose of 5.00 g/kg after 12 h of fasting (with enough water), respectively. The animals' condition were observed and recorded, as shown in table 1.

### 4. Results

No obvious symptoms of poisoning were observed in mice and no one died, which indicated that these compounds has no acute toxicity. Therefore, for the preparation of anti-HBV drugs, the compounds were of high value.

TABLE 1
The limited toxicity test

| | | | Number of | |
|---|---|---|---|---|
| | | Number of | dead | |
| Compounds | Dosage (g/kg) | animals | animals | Mortality % |
| A | 5.00 | 10 | 0 | 0 |
| B | 5.00 | 10 | 0 | 0 |
| C | 5.00 | 10 | 0 | 0 |
| D | 5.00 | 10 | 0 | 0 |
| E | 5.00 | 10 | 0 | 0 |
| F | 5.00 | 10 | 0 | 0 |
| G | 5.00 | 10 | 0 | 0 |

In conclusion, these compounds, with definite anti-HBV activity, high efficiency and low toxicity, have potential to be used for anti-HBV drug preparation, which provided a possibility for the screening and development of clinical medicine, and were of high value for application.

1. A method of treatment of hepatitis B comprising administering to a subject 15-substituted andrographolide derivative represented by formula 1, 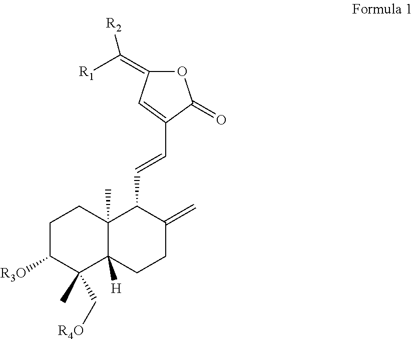
wherein R₁ is hydrogen; R₂ is phenyl, 4-fluorophenyl, 4-chlorophenyl, 4-bromophenyl, 3-fluorophenyl, 3-chlorophenyl, 3-bromophenyl, methoxyphenyl or polymethoxyphenyl; R₃ and R₄ are each independently hydrogen or COR₅, of which R₅ is 3-pyridyl; but when R₂ is 4-chlorophenyl, neither R₃ nor R₄ is COR₅.

2. The method according to claim 1, wherein R₁ is hydrogen; R₂ is phenyl, 4-fluorophenyl, 4-chlorophenyl, 4-bromophenyl, 3-fluorophenyl, 3-chlorophenyl, 3-bromophenyl, 3-methoxyphenyl or 4-methoxyphenyl; R₃ and R₄ are both hydrogen.

3. The method according to claim 1, wherein R₁ is hydrogen; R₂ is phenyl, 4-fluorophenyl, 4-bromophenyl, 3-fluorophenyl, 3-chlorophenyl, 3-bromophenyl, 3-methoxyphenyl or 4-methoxyphenyl; R₃ and R₄ are both COR₅, wherein R₅ is 3-pyridyl.

4. The method according to claim 1, wherein the 15-substituted andrographolide derivative is one of the following compounds:
A: R₁=H, R₂=4-Cl—C₆H₄, R₃=R₄=H;
B: R₁=H, R₂=C₆H₅, R₃=R₄=H;
C: R₁=H, R₂=4-F—C₆H₄, R₃=R₄=H;
D: R₁=H, R₂=4-F—C₆H₄, R₃=R₄=COR₅, R₅=C₅H₄N;
E: R₁=H, R₂=4-Br—C₆H₄, R₃=R₄=H;
F: R₁=H, R₂=3-Br—C₆H₄, R₃=R₄=COR₅, R₅=C₅H₄N;
G: R₁=H, R₂=2,4,5-triMeO—C₆H₂, R₃=R₄=H.

5. The method according to claim 1, wherein the 15-substituted andrographolide derivative is used alone as an active ingredient, or in combination with another active ingredient, and mixed with a pharmaceutically acceptable auxiliary and/or additive, in an oral or injectable preparation.

6. The method according to claim 5, wherein the oral preparation is a tablet, pill, capsule, granule or syrup; and wherein the injectable preparation is an injection or a lyophilized powder for injection.
